(19) United States
(12) Patent Application Publication
(10) Pub. No.: US 2012/0150217 A1
(43) Pub. Date: Jun. 14, 2012

(54) Proximally Actuated Surgical Instrument

(76) Inventors: Dyson W. Hickingbotham, (US); David E. Booth, (US)

(21) Appl. No.: 13/324,784

(22) Filed: Dec. 13, 2011

Related U.S. Application Data
(60) Provisional application No. 61/422,643, filed on Dec. 13, 2010.

(51) Int. Cl. A61B 17/28

(52) U.S. Cl. 606/206

(57) ABSTRACT

A surgical instrument for easy use of an operative tool. The surgical instrument includes a handle, a bushing tubing assembly, a pressure plate assembly, a cap, a ball ring assembly, and the operative tool. The bushing tubing assembly is connected to a distal end of the handle, while the pressure plate assembly is positioned at a proximal end of the handle and extends to a clamp disposed within the handle. The cap is positioned across from a proximal end of the pressure plate assembly. The ball ring assembly includes a plurality of balls and positioned between the pressure plate assembly and the cap. The operative tool is inserted through the bushing tubing assembly and the guide tube and then connects to the cap.

### CROSS-REFERENCE TO RELATED APPLICATIONS

This application is claims benefit of the filing date under 35 U.S.C. §119(e) of Provisional Patent Application No. 61/422,643, filed Dec. 13, 2010.

### FIELD OF THE INVENTION

The invention relates to surgical instrument and in particular to a proximally actuated ophthalmic surgical instrument.

### BACKGROUND

During ophthalmic surgical procedures, it is often necessary to dissect, cut delaminate or otherwise manipulate delicate tissues within the eye. Miniaturized instruments such as, for example forceps, scissors, and the like have been developed in order to allow a surgeon to operate on and within the patients eye. Known devices use plunger like components which are generally actuated by finger operated levers. More recently, handheld instruments have allowed for actuation of a distally mounted tool by application of radial force about the instruments applied around the circumference. These instruments are known as being omniactuable. Examples of such instruments are shown in U.S. Pat. No. 6,488,695 and U.S. Pat. No. 6,391,046 wherein circumferential actuators are radially displaceable in response to compression forces to operate a distally mounted tool. There is a need, however for a more simple, inexpensive actuator handle for such tools which provides smoother and effortless actuating mechanism motion.

### SUMMARY

Accordingly, the invention was devised in light of the problems described above, the invention is a proximally actuated surgical instrument provide a technician or practitioner easy use of an operative tool disposed at a distal end of the instrument. Specifically, the surgical instrument includes a handle, a bushing tubing assembly, a pressure plate assembly, a cap, a ball ring assembly, and the operative tool. The bushing tubing assembly is connected to a distal end of the handle, while the pressure plate assembly is positioned at a proximal end of the handle and extends to a clamp disposed within the handle. The cap is positioned across from a proximal end of the pressure plate assembly. The ball ring assembly includes a plurality of balls and positioned between the pressure plate assembly and the cap. The operative tool is inserted through the bushing tubing assembly and the guide tube and then connects to the cap.

### BRIEF DESCRIPTION OF THE DRAWINGS

The invention will now be described by way of example with reference to the accompanying figures of which:

FIG. 1 is a perspective view of the surgical instrument according to the invention;

FIG. 2 is a cross sectional view taken along the line 2-2 of FIG. 1;

FIG. 3 is an exploded side view of the instrument of FIG. 1;

FIG. 4 is a perspective view of an insert used in instrument of FIG. 1;

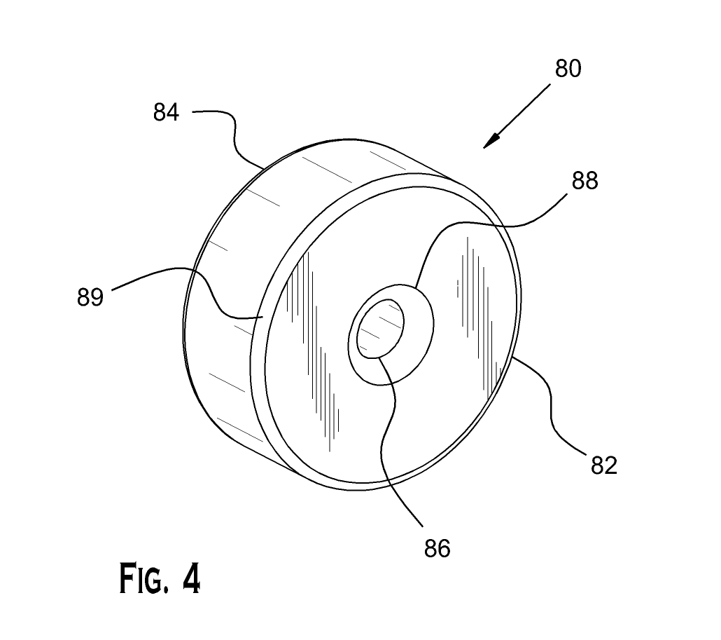

FIG. 5 is a perspective view of a clamp used in the instrument of FIG. 1; and

FIG. 6 is an exploded perspective view of the proximal end of the instrument of FIG. 1.

### DETAILED DESCRIPTION OF THE EMBODIMENT(S)

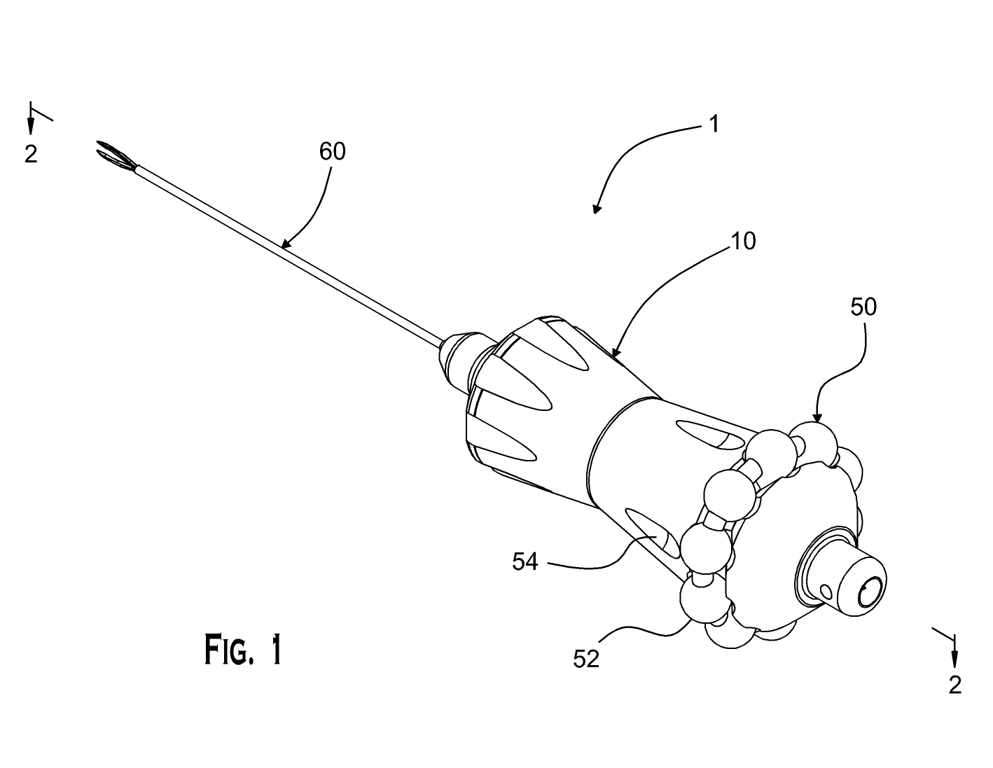

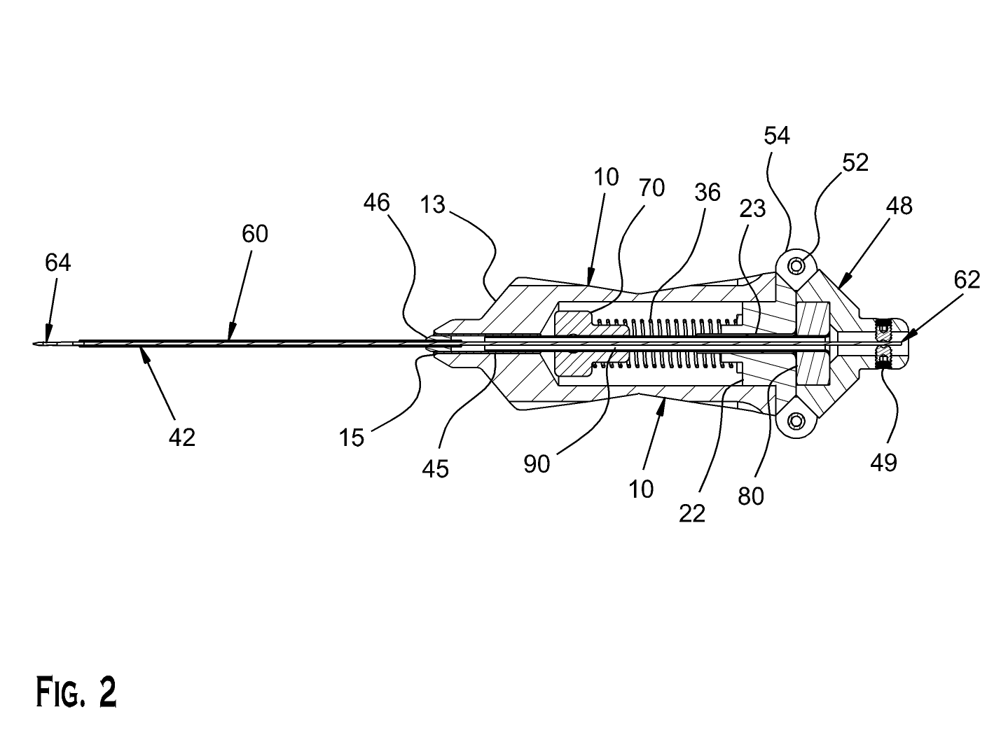

The invention will now be described in greater detail with reference to the embodiment shown in the accompanying figures. First, with reference to FIGS. 1 and 2, major components of the instrument 1 include a handle 10, a pressure plate assembly 20, a bushing tubing assembly 40, a tip sleeve 44, a ball ring assembly 50, a clamp 70, a spring 36, a spacer 34, an insert 80, a guide tube 90, and an operative tool 60, which in this case is shown as jaws.

Now each of the major components will be described in greater detail. First, a handle 10 is a generally hollow cylindrical component having a profile which is suitable for grasping by a surgeon. In this case, its profile begins at a proximal end 12 with a proximal diameter. At the proximal end 12, a series of bearing surfaces 17 are located around the circumference thereof. The bearing surfaces 17 shown in this embodiment are shaped as semi-spherical concavities to receive balls 52 and it should be understood by those reasonably skilled in the art that the shape of the bearing surfaces 17 should be selected to accommodate the component against which it bears, in this case a ball 52. Other shapes for the bearing surfaces 17 are therefore contemplated and within the scope of the invention. Moving from the proximal end toward the distal end 16, the profile first tapers inward to a smaller proximal area 14. From its narrowest point, in the smaller proximal area 14, the profile continues toward the distal end 16 in a widening fashion wherein gripping features 18 are provided around the circumferential profile. Moving toward the distal end, the profile tapers again inward from a transition 19 adjacent to gripping features 18.

The gripping features 18, in this embodiment are shown as a series of concavities spaced apart from each other around the periphery of a handle 10 in near the transition 19. It should be understood by those reasonably skilled in the art that although these gripping features 18 are shown here as a series of concavities, they may take other shapes such as depressions, or projections of various shapes and may alternately be formed of different materials which facilitate hand gripping by the surgeon. At the transition 19 the handle profile begins to take a generally conical shape moving toward its distal end 16. This generally conical shape profile has a tip 13 through which an opening 15 passes to the distal end 16. In this embodiment, the handle 10 is molded from a plastic material, however, it should be understood by those reasonably skilled in the art that other suitable materials, molding and/or machining techniques may be employed to fabricate a handle 10.

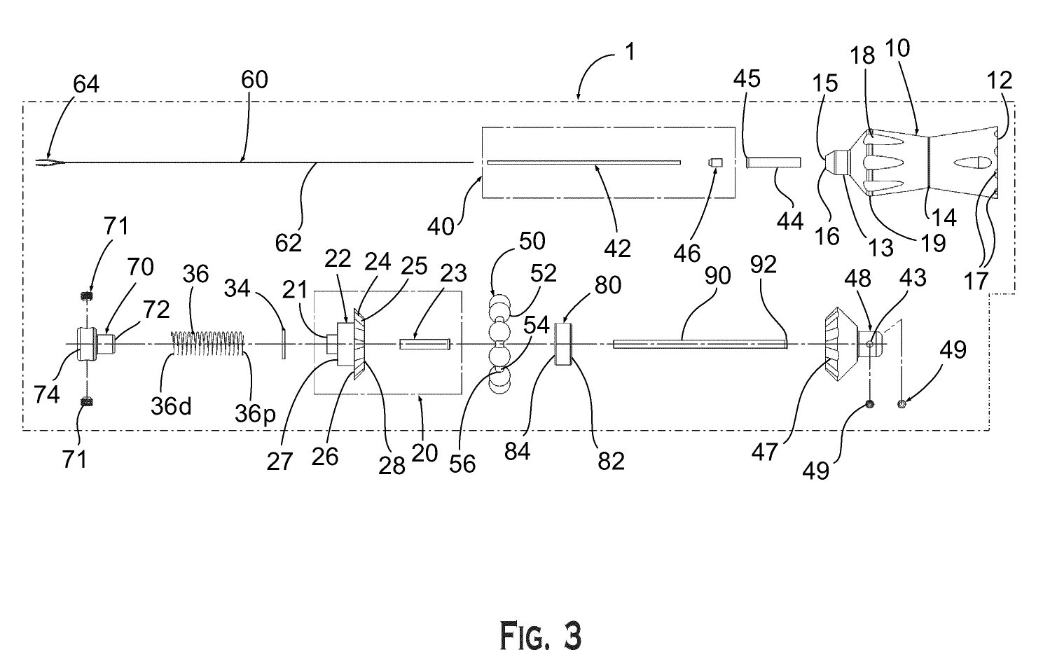

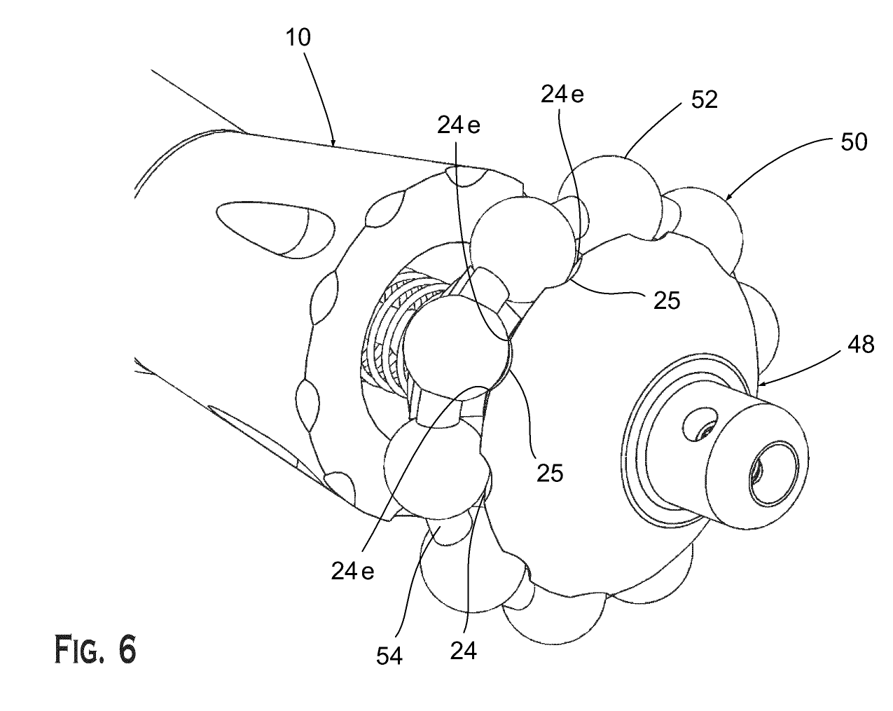

Next, referring to FIGS. 3 and 6, the pressure plate assembly 20 will be described in greater detail. The pressure plate assembly 20 consists of a pressure plate 22, and a pressure plate tube 23. The pressure plate 22 is a generally cylindrical component, formed from plastic or another suitable material, having a series of primary bearing surfaces 24 arranged along an integral flange 26 positioned along and angled toward the proximal end 28. The primary bearing surfaces 24 are designed to have concavities 25 around the circumference thereof. In this embodiment, the concavities 25 are generally U-shaped channels and are designed to receive the balls 52 along edges 24e thereof. Such edges 24e act as rails upon which the balls 52 ride. Therefore, the concavities 25 may take other shapes which are suitable for receiving and easily gliding the balls 52 as will be described below. It should be understood by those reasonably skilled in the art that the shape of the primary bearing surfaces 24 should be selected to accommodate the component against which it bears, in this case a ball 52. Other shapes for the primary bearing surfaces 24 are therefore contemplated and within the scope of the invention. In this embodiment, the pressure plate 22 may be formed by molding or machining any suitable plastic or metallic material. On its distal end, the pressure plate 22 has a circumferential spring shoulder 27 and a narrow circumferential projection 21 projecting distally from the circumferential spring shoulder 27.

The spring 36 is formed as a coil spring and is positioned adjacent to the pressure plate 22 and against a spacer 34 on its proximal end 36p, while its distal end 36d is positioned against a clamp 70 which will be described in greater detail below. In this embodiment, the spacer 34 which takes the form of a flat washer is located between the proximal end 36p of the spring 36 and the pressure plate 22.

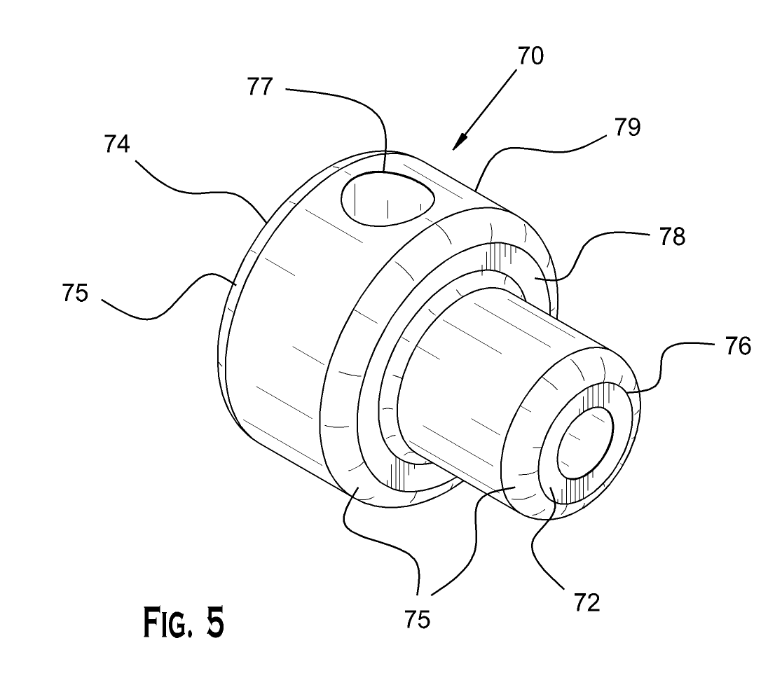

Referring now to FIGS. 5 and 3, the clamp 70 will be described in greater detail. The clamp 70 is generally cylindrically shaped and extends from a proximal end 72 to a distal end 74. A central passageway 76 passes from the proximal end 72 to the distal and 74. A wide portion 79 is formed near the distal end 74. A pair of threaded openings 77 pass from an outer surface of the wide portion 79 to the central passageway 76. Set screws 71 are threadingly received inside each opening 77. A flange 78 is provided at the proximal end of the wide portion 79 and rounded surfaces 75 are located around proximal end 72, distal end 74 and flange 78. In this embodiment, the clamp 70 is formed of the same material as the pressure plate 22 but it should be understood by those reasonably skilled in the art that other suitable materials for clamping the spring 36 and for receiving set screws 71 may be substituted.

Referring now to FIGS. 5 and 3, the insert 80 will be described in greater detail. The insert 80 is a generally cylindrical component having a proximal end 82 and a distal end 84 through which a guide tube receiving passageway 86 passes. A tapered surface 88 is provided around both ends of the guide tube receiving passageway 86. Rounded surfaces 89 are located around the periphery of the proximal and distal ends 82, 84.

The guide tube 90 is a generally cylindrical tubular component having a flared proximal end 92. The outer diameter of the guide tube 90 is selected to snugly fit within the guide tube receiving passageway 86 of the insert 80 and slidingly fit within the pressure plate tube 23, the central passageway 76 of the clamp 70, and the tip sleeve 44. The inner diameter of the guide tube 90 is selected to pass the rod 62 with a clearance therebetween.

Next, the ball ring assembly 50 will be described in greater detail. The ball ring assembly 50 consists of a plurality of balls 52 and a ring 54. Each of the balls 52, is a generally spherical component having a generally cylindrical passageway 56 formed therein. The passageway 56 may take a cylindrical shape or may alternatively be formed to have a slight arcuate shape. In this embodiment, the balls 52 are made of a plastic material, however, they may be formed of other suitable materials such as metal, glass, Teflon or any other material which glides freely between the bearing surfaces 24, 47. The ring 54 is made of a flexible material such as silicone tubing but may be made of any other suitable flexible tubular material. In this embodiment, the ring 54 has a circular cross section but it should be understood by those reasonably skilled in the art that the cross-section may be modified and adapted to various different passageway shapes. For example, the ring 54 may have a square or rectangular cross-section. The ball ring assembly 50 is assembled by threading the ring 54 through each of the passageways 56 and then joining by bonding, fusing or any other suitable method the respective ends of the ring 54 together. Once assembled, the ball ring assembly 50 remains somewhat flexible or stretchable.

The tip sleeve 44 is a generally tubular component having a flared distal end 45. The tip sleeve 44, in this embodiment, is formed of stainless steel, however, it should be appreciated by those reasonably skilled in the art that other suitable materials for forming an appropriate snug fit between the tip bushing 46, the tool tube 42, and the tip 13 may be utilized. The diameter of the tip sleeve 44 is selected so that its outer diameter fits snugly within the opening 15 while its inner diameter snugly receives the tip bushing 46 at its distal end and also receives, at its proximal end, the guide to 90 which will be described in greater detail below.

Turning now to the bushing tubing assembly 40, it consists of a tool tube 42, and a tip bushing 46. The tool tube 42, in this embodiment, formed of a stainless steel material, has a diameter sized for slidingly receiving the tool rod 62 which will be described in greater detail below. The tool tube 42 is designed to fit snugly within the tip bushing 46. The snug fit may be achieved by an interference or these parts may be otherwise attached by, for example an adhesive or other bonded connection. The tip bushing 46 is sized to fit snugly within the tip sleeve 44 which fits into the opening 15 of the tip 13. Likewise, the snug fit may be achieved by interference or these parts may be otherwise attached by, for example an adhesive or other bonded connection. In this embodiment, the tip bushing 46 is made of a stainless steel material, however, it should be understood that the tip bushing 46 may be made of other suitable materials that form an interference fit between the inner and tool tube 42 and tip sleeve 44.

The cap 48 has a generally conical shape and has an opening passing through its center from a distal end to a proximal end thereof. Along the distal end, a plurality of bearing surfaces 47 are located and are similarly shaped to primary bearing surfaces 24 such that they form a pair of rails for each ball 52 to ride upon. The bearing surfaces 47 face toward the pressure plate assembly 20. Near its proximal end, it has a pair of threaded lateral openings 43 passing from its outer circumference into the opening passing through its center from the distal to proximal ends. A pair of set screws 49 are threadingly inserted into the threaded lateral openings 43. The cap 48, in this embodiment, is formed of the same material as the pressure plate 22 but it should be understood by those reasonably skilled in the art that it may be formed of any suitable molded or machined material.

The operative tool 60 consists of a rod 62 having a tool 64 integrally formed on its distal end. In this embodiment, the tool 64 is a set of opposing jaws designed to open and close upon actuation as will be more fully described below. The rod 62 has a diameter and is profiled to slidingly fit within the tool tube 42. The operative tool 60 is made of a suitable surgical metal, however it should be understood by those reasonably skilled in the art that other materials suitable for a selected tool 64 may be substituted.

Now, assembly of the instrument 1 will be described in greater detail. The pressure plate assembly 20 is completed by inserting the pressure plate tube 23 into the pressure plate 22. Likewise, the bushing tubing assembly 40 is completed by inserting the tool tube 42 into the tip bushing 46 which is then inserted into the flared end 45 of the tip sleeve 44. The proximal end of the tip sleeve 44 is then inserted into the opening 15 of the tip 13. The guide tube 90 is fit and secured into the guide tube receiving passageway 86 of the insert 80. The assembled guide tube 90 and insert 80 are inserted into the distal end of the cap 48. With the ball ring assembly 50 position over its proximal end 28, the pressure plate 22, spacer 34, spring 36, and clamp 70 are slid over the guide tube 90 from its distal end. Set screws 71 are driven in to fix the clamp 70 to the guide tube 90. The handle 10 is then positioned over the clamp 70 and the pressure plate 22. Finally, the rod 62 is inserted into the tool tube 42 and passes through the guide tube 90 to the proximal end of the cap 48 where it is secured by set screws 49.

Now, operation of the instrument 1 will be described in greater detail. Referring to FIG. 2, the instrument 1 is shown in its unactuated position wherein the tool 64 protrudes from the tool tube 42. The instrument 1 is actuated by depressing the balls 52 of the ball ring assembly 50. This action applies an axial force to the bearing surfaces 47, 24 urging cap 48 and pressure plate assembly 20 apart from each other. The guide tube 90, clamp 70, and rod 62 move in unison with the cap 48 against the spring 36 and pressure plate 22 while the guide tube 90 slides within pressure plate tube 23 and tip sleeve 44. The tool 64 is thereby retracted into the tool tube 42 against the spring 36 bias. Releasing depression of the ball ring assembly 50 causes the cap 48 to move back towards the distal end under the spring force thereby causing the tool 64 to protrude from the tool tube 42 in a controlled manner.

It should be understood that while the invention has been described here in view of an exemplary embodiment, other embodiments having variations on the concepts are contemplated and intended to be within the scope of the invention.

What is claimed is:

1. A surgical instrument comprising:
a handle;
a bushing tubing assembly connected to a distal end of the handle;
a pressure plate assembly positioned at a proximal end of the handle and extending to a clamp disposed within the handle;
a cap positioned across from a proximal end of and corresponding with the pressure plate assembly;
a ball ring assembly having a plurality of balls and positioned between the pressure plate assembly and the cap; and
an operative tool inserted through the bushing tubing assembly and connecting to the cap.

2. The surgical instrument according to claim 1, further comprising a spring disposed between the clamp and the pressure plate assembly.

3. The surgical instrument according to claim 2, further comprising a guide tube extending from the clamp to the pressure plate assembly;

4. The surgical instrument according to claim 3, further comprising an insert positioned between the pressure plate assembly and the cap.

5. The surgical instrument according to claim 4, wherein the insert is a cylindrical component having a guide tube receiving passageway through which the guide tube is inserted.

6. The surgical instrument according to claim 3, wherein the pressure plate assembly includes a pressure plate and a pressure plate tube inserted into the pressure plate.

7. The surgical instrument according to claim 6, wherein the pressure plate includes a plurality of primary bearing surfaces arranged along an integral flange positioned along and angled toward a proximal end.

8. The surgical instrument according to claim 7, wherein the plurality of primary bearing surfaces are concavities for receiving and engaging the plurality of balls.

9. The surgical instrument according to claim 8, wherein the plurality of primary bearing surfaces are positioned to face the cap.

10. The surgical instrument according to claim 6, wherein a proximal end of the spring is positioned adjacent to the pressure plate and a distal end of the spring is positioned against the clamp.

11. The surgical instrument according to claim 6, wherein the guide tube is tubular having an outer diameter that snugly fits within the pressure plate tube, and the clamp.

12. The surgical instrument according to claim 3, wherein the clamp includes a flange and a central passageway that passes from a proximal end to a distal end.

13. The surgical instrument according to claim 12, wherein the clamp includes a further comprising a threaded opening for receiving a set screw to secure the guide tube.

14. The surgical instrument according to claim 1, wherein the cap is conical and includes an opening passing through a center.

15. The surgical instrument according to claim 14, wherein the cap includes a plurality of secondary bearing surfaces for receiving and engaging the plurality of balls to ride upon.

16. The surgical instrument according to claim 15, wherein the plurality of secondary bearing surfaces are positioned to face the pressure plate assembly.

17. The surgical instrument according to claim 16, further comprising a set screw received through the cap to secure the operative tool.

18. The surgical instrument according to claim 1, wherein the ball ring assembly includes a flexible ring.

19. The surgical instrument according to claim 18, wherein each ball of the plurality of balls is a spherical component having a cylindrical passageway formed therein.

20. The surgical instrument according to claim 19, wherein the ball ring assembly is assembled by threading the flexible ring through the cylindrical passageway of each ball of the ball ring assembly and then joining respective ends of the flexible ring together.

21. The surgical instrument according to claim 1, further comprising a tip sleeve having (1) an outer diameter that fits snugly within an opening of the handle and (2) an inner diameter that snugly receives the bushing tubing assembly.

22. The surgical instrument according to claim 1, wherein the bushing tubing assembly includes (1) a tool tube for slidingly receiving the operative tool and (2) a tip bushing that snugly fits within a tip sleeve received in an opening of the handle.

23. The surgical instrument according to claim 22, wherein the tool tube is inserted into the tip bushing and then inserted into a flared end of the tip sleeve.

24. The surgical instrument according to claim 1, wherein the operative tool includes a rod having a tool integrally formed on a distal end.

25. The surgical instrument according to claim 24, wherein the tool is a set of opposing jaws that open and close upon actuation of the ball ring assembly.

26. The surgical instrument according to claim 25, wherein the rod slidingly fits within the bushing tubing assembly.
